(19) United States
(12) Patent Application Publication
(10) Pub. No.: US 2015/0290051 A1
(43) Pub. Date: Oct. 15, 2015

(54) WEARABLE MALE INCONTINENCE PROTECTOR

(71) Applicant: Brendan Gierer, Wright City, MO (US)

(72) Inventor: Brendan Gierer, Wright City, MO (US)

(21) Appl. No.: 14/684,543

(22) Filed: Apr. 13, 2015

Related U.S. Application Data
(60) Provisional application No. 61/978,307, filed on Apr. 11, 2014.

(51) Int. Cl. A61F 13/56; A61F 13/471

(52) U.S. Cl. CPC A61F 13/5605; A61F 13/471; A61F 2013/15146

(57) ABSTRACT

A wearable tube apparatus for placement over the genitals of a male subject to absorb urine released from the penis and wick moisture from the genitals, thereby preventing the subject from soiling himself The wearable tube apparatus comprises an elongated tube body defined by a flexible, hollow tubular member with a fluid resistant outer layer and a fluid absorbent inner layer. The flexible tube body includes a closeable opening on its superior region and an elastic opening on its proximal end and is structured to taper upward towards the proximal end. The closeable opening enables a caregiver or the user to reach into the hollow area of the elongated tube body so adjust the genitals or so the interior absorbent layer can be touched to check for moisture without requiring the removal of the wearable tube apparatus.

### CROSS REFERENCE TO RELATED APPLICATIONS

This application claims the benefit of and incorporates by reference co-pending U.S. provisional patent application Ser. No. 61/978,307 filed Apr. 11, 2014.

### BACKGROUND OF THE INVENTION

1. Field of the Invention

This invention relates generally to incontinence accessories and, more particularly, to a wearable tube apparatus for a man which absorbs urine to prevent a user from soiling himself with urine.

2. Description of the Prior Art

It is well established that as people age or are infirmed, many male patients suffer from urinary incontinence. Diapers are often worn to absorb urine under such circumstances and are known to adequately absorb urine. But a problem which still exists, is that diapers on a full grown man are often difficult to change and have to be changed frequently to prevent the occurrence of skin problems or ulcerations in pressure areas, such as the coccyx. Thus, there remains a need for a wearable tube apparatus which can be placed over the wearer's genitals and absorb urine released from the same. It would be helpful if such a wearable tube apparatus was sized to enable it to be changed. by one caregiver. It would be additionally desirable for such a wearable tube apparatus to include a closeable opening to enable caregivers to assist with placement of the male genitals in the apparatus and to monitor wetness. It is further desirable for such a wearable tube apparatus to be structured to keep the skin over pressure areas, such as the coccyx, dry.

The Applicant's invention described herein provides for a tube apparatus adapted to be worn by a male user over his genitals and absorb urine released therefrom. The primary components in Applicant's wearable tube apparatus are a tube having an absorbed inner material and a fluid resistant outer material. When in operation, the wearable tube apparatus enables a male patient to wear a protective structure which is less bulky and easier to change than a diaper. As a result, many of the limitations imposed by prior art structures are removed.

### SUMMARY OF THE INVENTION

A wearable tube apparatus for placement over the genitals of a male subject to absorb urine released from the penis and wick moisture from the genitals, thereby preventing the subject from soiling himself. The wearable tube apparatus comprises an elongated tube body defined by a flexible, hollow tubular member with a fluid resistant outer layer and a fluid absorbent inner layer. The flexible tube body includes a closeable opening on its superior region and an elastic opening on its proximal end and is structured to taper upward towards the proximal end. The closeable opening enables a caregiver or the user to reach into the hollow area of the elongated tube body so adjust the genitals or so the interior absorbent layer can be touched to check for moisture without requiring the removal of the wearable tube apparatus.

It is an object of this invention to provide a wearable tube apparatus which can be placed over the wearer's genitals and absorb urine released from the same.

It is another object of this invention to provide a wearable tube apparatus sized to enable it to he changed by one caregiver.

It is yet another object of this invention to provide a wearable tube apparatus that includes a closeable opening that enables caregivers to assist with placement of the male genitals in the apparatus and to monitor wetness.

It is still another object of this invention to provide a wearable tube apparatus structured to keep the skin over pressure areas, such as the coccyx, dry.

These and other objects will be apparent to one of skill in the art.

### BRIEF DESCRIPTION OF THE DRAWINGS

FIG. 1 is a side perspective view of a wearable tube apparatus built in accordance with the present invention.

### DETAILED DESCRIPTION OF THE INVENTION

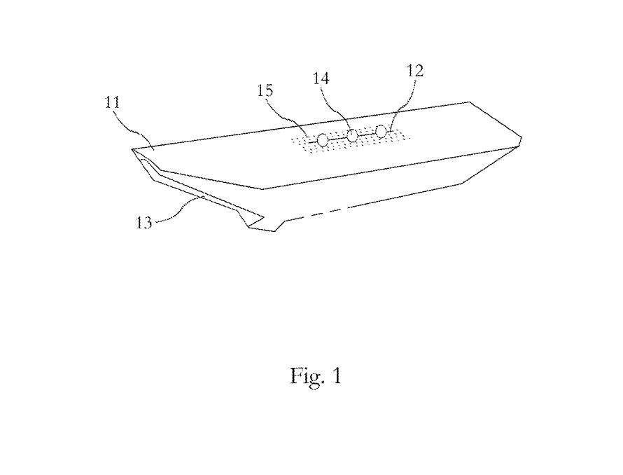

Referring now to the drawings and in particular FIG. 1, a wearable tube apparatus 10 is shown having an elongated tube body 11 having a closeable opening on its superior region and an elastic opening 13 on its proximal end. The elongated tube body 11 is defined by a flexible, hollow tubular member with a fluid resistant outer layer and a fluid absorbent inner layer. Running from the distal end to the proximal end, the elongated tube body 11 is structured to taper upward towards the proximal end. In the preferred embodiment, the elongated tube body 111 measures 10 inches long and has an average diameter of 4 inches.

The closeable opening 12 includes a plurality of fastener pieces 14 which can be releasably secured over the closeable opening 12 to hold the closeable opening in a closed position. The fastener pieces 14 can also be lifted off the closeable opening to enable the closeable opening to be placed in an open position. In the preferred embodiment, the fastener pieces 14 are defined by sections of a hook and loop fastener fabric, with a corresponding fastener engaging section 15 permanently attached to or otherwise disposed on the elongated tube body 11 around the surface of the closeable opening 12. When in the open position, the closeable opening 12 is sized to enable a caregiver to reach a gloved hand into the hollow area of the elongated tube body 11 so that the penis and/or scrotum of the wearer can be adjusted or so the interior absorbent layer can be touched to check for moisture.

The elastic opening 13 is defined by a band of elastic material which lines the exterior of the opening. The beyond the elastic band, the material around the elastic opening includes perforations to enhance its ability to be deformed to stretch over a wearer's genitals. In use, manual pressure in an outward direction is applied to the elastic opening 13 to stretch it outward and enable the elongated tube body 11 to be slid over the wearer's penis and scrotum. Once the elongated tube body 11 is in place on the wearer, with the elastic opening 13 contacting the wearer's groin area, the manual pressure is removed, allowing the elastic opening 13 to return to a smaller size that enables it to remain in place over the wearer's penis and scrotum.

It is appreciated that when in place on a wearer's genitals, the elastic opening 13 and tube body 11 are together shaped such that the mass of the wearer's scrotum acts to hold the wearable tube apparatus 10 in place without the use of an adhesive.

In an alternate embodiment, a built in fluid measure device is included in the hollow portion of the elongated tube body to enable urinary output to be measured.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A wearable tube apparatus for placement of a male subject, comprising:
an elongated tube body defining a flexible, hollow tubular member with a proximal end and a distal end and including a closeable opening on the tubular surface between the proximal end and distal end;
said proximal end including an opening sized to receive the genitals of a male subject such that the subject's genitals can be positioned in and encased in the tube body; and
said closeable opening configured to provide access inside the hollow tubular member while the wearable tube apparatus is in place on a subject.

2. The wearable tube apparatus of claim 1, wherein said proximal end and tube body are configured such that the mass of a subject's scrotum acts to hold the wearable tube apparatus in place without the use of an adhesive.

3. The wearable tube apparatus of claim 1, wherein said proximal end defines an elastic rim adapted to be stretched outwardly when a subject's genitals are moved into the tube body.

4. The wearable tube apparatus of claim 1, wherein the closeable opening is disposed on a superior region of the tube body.

5. The wearable tube apparatus of claim 1, additionally comprising at least one fastener piece adapted to hold the closeable opening closed.

6. The wearable tube apparatus of claim 5, wherein the fastener piece is removable.

7. The wearable tube apparatus of claim 5, wherein the closeable opening includes a fastener engaging section adjacent thereto and said at least one fastener pieces are adapted to releasably engage the fastener engaging section.

8. The wearable tube apparatus of claim 1, wherein said elongated tube body includes a fluid resistant outer layer and a fluid absorbent inner layer.

9. The wearable tube apparatus of claim 1, wherein said elongated tube body is structured to taper upward moving towards the proximal end.

10. A wearable tube apparatus for placement of a male subject, comprising:
an elongated tube body defining a flexible, hollow tubular member with a proximal end and a distal end and including a closeable opening on the tubular surface between the proximal end and distal end;
said proximal end including an opening with an elastic rim that is sized to receive the genitals of a male subject such that the subject's genitals can be positioned in and encased in the tube body,
said proximal end and tube body configured such that the mass of a subject's scrotum acts to hold the wearable tube apparatus in place without the use of an adhesive;
said closeable opening configured to provide access inside the hollow tubular member while the wearable tube apparatus is in place on a subject; and
said elongated tube body having a fluid resistant outer layer and a fluid absorbent inner layer.

11. The wearable tube apparatus of claim 10, additionally comprising at least one fastener piece adapted to hold the closeable opening closed, wherein the closeable opening includes a fastener engaging section adjacent thereto and said at least one fastener pieces are adapted to releasably engage the fastener engaging section.
